(19) United States
(12) Patent Application Publication
(10) Pub. No.: US 2013/0046082 A1
(43) Pub. Date: Feb. 21, 2013

(54) POLAR ORGANIC EXTRACT OF EURYCOMA LONGIFOLIA

(71) Applicants: Kit Lam CHAN, Penang (MY); Bin Seng Low, Penang (MY); David Sue San Ho, Ipoh (MY)

(72) Inventors: Kit Lam CHAN, Penang (MY); Bin Seng Low, Penang (MY); David Sue San Ho, Ipoh (MY)

(73) Assignee: Universiti Sains Malaysia, Pinang (MY)

(21) Appl. No.: 13/494,945

(22) Filed: Jun. 12, 2012

Related U.S. Application Data
(62) Division of application No. 12/302,875, filed on Nov. 28, 2008.

(30) Foreign Application Priority Data
Aug. 7, 2006 (MY) PI 20063783

(51) Int. Cl. C07D 493/08; C07D 311/10; C07H 17/075; C07H 15/26

(52) U.S. Cl. 536/18.1; 549/290; 549/275

(57) ABSTRACT

The present invention provides a composition including a polar organic extract of Eurycoma longifolia and a fraction derived from the polar organic extract, said composition comprising of quassinoids, coumarins, their glycosides, analogues and derivatives, which exhibits bioactivity of increasing spermatozoa production and spermatozoa quality in terms of morphology and motility, as well as increasing testosterone synthesis and release from cells of males. The extraction method of E. longifolia plant to produce the polar organic extract, and the subsequent purification to produce the fraction of polar organic extract containing the quassinoids, coumarins, their glycosides, analogues and derivatives, and uses for manufacturing a preparation for infertility treatment are also provided. The fraction of polar organic extract containing the quassinoids, coumarins, their glycosides, analogues and derivatives is formulated for medical applications via several routes of administration.

The present invention refers to the field of herbal medicine in particular, to a herbal pharmaceutical preparation for the treatment of male infertility or sexual dysfunction. More specifically, the present invention, relates to the composition of a polar organic extract of Eurycoma longifolia and a fraction thereof having bioactivities to improve spermatogenesis in males, the use for manufacturing a preparation for infertility treatment, and the extraction and purification methods thereof.

### BACKGROUND TO THE INVENTION

The roots or stems of Eurycoma longifolia Jack, also known traditionally as tongkat ali, penawar pahit, bedara pahit, tongkat baginda, petala bumi, pasak bumi, setunjang bumi, cay ba binh and plaa-lai-pueak, have been used in traditional and folk medicine either as a single herb or as part of multiple herb ingredients to treat dysentery, fever, malaria and sexual problems including male infertility. Numerous scientific papers have reported that the quassinoids derived from the plant extracts possessed antimalarial (Chan et. al., 1986; Kuo at al., 2004; Jiwajinda at al., 2002; Chan et al., 2004), antischistosomal (Jiwajinda et al., 2002), antiulcer (Tada et. al., 1991), antipyretic (Chan et. al., 1995), cytotoxic (Morita et. al. 1993; Kuo et al., 2004), antitumour promoting (Jiwajinda at al., 2002) and aphrodisiac activities (Ang and Sim, 1998).

Infertility in couples has been defined as the inability of the female to conceive after more than a year of unprotected sexual intercourse and is a major problem for those who are very keen to have children. According to WHO (2003), about 10-15% of over 186 million couples are affected by infertility, where the male has contributed to approximately 30-40% towards the problem (Royle and Walsh, 1992), and for 20-30% of the time, it may be a combination of both partners. .

Male infertility may be due to a lack of spermatozoa in the semen, deformed or structurally abnormal spermatozoa, lacking of motility to fertilize a female egg, genetic or endocrine disorder. Over 90% of the male infertility problems have been due to low spermatozoa count or poor quality spermatozoa (Winston, 1986).

A single spermatozoon is really all that is necessary for pregnancy but the chances of fertilization is reduced when there are less than 20 million motile spermatozoa in the semen sample. In some cases of male infertility, the problem may be overcome by simply increasing the number of spermatozoa. Presently, there is no publicly known method for increasing the spermatozoa count. Hence, a product assisting infertile males to increase their low spermatozoa count will potentially improve their fertility and bring tremendous benefits to infertile couples.

Eurycoma longifolia Jack has been reputed in Malay traditional remedy to increase male virility and has been documented to display aphrodisiac property (Zakaria and Ali Mohd, 1994).

United States patent application no. 20040087493 (Sambandan et al., 2004) broadly claimed that an aqueous extract of E. longifolia, comprising a glycopeptide with a molecular weight of 4,300 daltons and having between 30 and 39 amino acids and sugar residues, has activity of increasing testosterone synthesis, increasing testosterone release from cells, increasing sperm count and increasing sperm motility. The composition of this extract is also claimed for the treatment of sexual dysfunction or male infertility.

Unlike the prior art, it is an advantage of the present invention to provide a polar organic extract of Eurycoma longifolia and a fraction thereof comprising a composition of quassinoids, coumarins, their glycosides, analogues and derivatives, and having the activity of increasing spermatozoa production and increasing spermatozoa motility, as well as increasing testosterone synthesis and release from cells of males. The use of the composition for manufacturing a preparation for infertility treatment, the process of extract and fraction development, as well as isolation and identification of the chemical constituents are also provided in the present invention.

### SUMMARY OF THE INVENTION

The present invention broadly discloses the composition of a polar organic extract of Eurycoma longifolia and a fraction derived from the polar organic extract, said composition comprising of quassinoids, coumarins, their glycosides, analogues and derivatives, which exhibits bioactivity of increasing spermatozoa production and spermatozoa quality in terms of morphology and motility, as well as increasing testosterone synthesis and release from cells of males. The extraction method of E. longifolia plant to produce the polar organic extract, and the subsequent purification to produce a fraction of polar organic extract containing the quassinoids, coumarins, their glycosides, analogues and derivatives, and their uses for manufacturing a preparation for infertility treatment are also provided. The fraction of polar organic extract containing the quassinoids, coumarins, their glycosides, analogues and derivatives is formulated for medical applications via several routes of administration.

According to one aspect of the present invention, there is provided a composition including a polar organic extract of Eurycoma longifolia, the extract comprises quassinoids, coumarins, their glycosides, analogues and derivatives, and having a percentage by weight of up to 5%.

The polar organic extract has a biological activity that includes increasing spermatozoa count, increasing spermatozoa motility, as well as increasing testosterone synthesis and release from cells of males.

According to another aspect of the present invention, there is provided a composition including a fraction of a polar organic extract of Eurycoma longifolia, the fraction having a percentage by weight of 10% to 25% of the polar organic extract and comprises:

quassinoids, which include eurycomanone, 13α,21-dihydroeurycomanone, 13(21)-epoxyeurycomanone, eurycomanol and its glycoside, eurycomaoside and its aglycone, including all their analogues and derivatives; and

coumarins, which include 6-methoxycoumarin-7-O-α-D-glycopyranoside, its other glycosides, analogues and derivatives.

The fraction of polar organic extract of Eurycoma longifolia has a biological activity that includes increasing spermatozoa count, increasing spermatozoa motility, as well as increasing testosterone synthesis and release from cells of males.

According to still another aspect of the present invention, there is provided a method for isolating the bioactive components from Eurycoma longifolia, which includes:

preparing a polar organic extract from Eurycoma longifolia plant materials;

subjecting the polar organic extract to fractionation through a styrene-divinylbenzene synthetic resin or a dextran synthetic resin and eluted sequentially with water, a mixed solution of water and organic solvent, and an organic solvent to obtain a fraction of a polar organic extract of Eurycoma longifolia; and

isolating and purifying the bioactive components by partition, chromatographic and recrystallization methods.

The polar organic extract is prepared by subjecting pulverized roots, barks or stems of Eurycoma longifolia to extraction with an organic solvent or a mixed solution of water and organic solvent thereof.

It is preferred that the adsorbent resin-packed chromatographic column used for the fractionation of the polar organic extract to the selected fraction is a styrene-divinylbenzene synthetic resin or a dextran synthetic resin.

It is further preferred that the organic solvent is a lower alcohol or a polar organic solvent that can be selected from the group consisting of methanol, ethanol, isopropyl alcohol and acetone.

The fraction of polar organic extract of Eurycoma longifolia obtained by the method of the present invention contains quassinoids, which include eurycomanone, 13α,21-dihydroeurycomanone, 13(21)-epoxyeurycomanone, eurycomanol and its glycoside, eurycomaoside and its aglycone, including all their analogues and derivatives; and coumarins, which include 6-methoxycoumarin-7-O-α-D-glycopyranoside, its other glycosides, analogues and derivatives, and exhibits bioactivity of increasing spermatozoa count, increasing spermatozoa motility, as well as increasing testosterone synthesis and release from cells of males.

The present invention includes isolation and identification of the components of the fraction of a polar organic extract of Eurycoma longifolia, consisting of quassinoids comprising eurycomanone, 13α,21-dihydroeurycomanone, 13(21)-epoxyeurycomanone, eurycomanol and its glycoside, eurycomaoside and its aglycone, including all their analogues and derivatives; and coumarins comprising 6-methoxycoumarin-7-O-α-D-glycopyranoside, it is other glycosides, analogues and derivatives, for increasing spermatozoa count, increasing spermatozoa motility, as well as increasing testosterone synthesis and release from cells of males. These quassinoids and coumarins including their analogues and derivatives are analyzed by chromatographic processes including reversed phase high-performance liquid chromatography (HPLC) and mass spectroscopy (MS) and identified by ultraviolet, infrared, mass spectroscopies and nuclear magnetic resonance and X-ray diffraction analysis.

According to yet another aspect of the present invention, pharmaceutical preparations are provided. The preparations include an effective amount of the foregoing composition of a polar organic extract of Eurycoma longifolia and/or a fraction derived from the polar organic extract, along with a pharmaceutically acceptable carrier useful In treating sexual dysfunction or infertile males by improving spermatogenesis, which increases spermatozoa count, spermatozoa motility, testosterone synthesis and increasing testosterone release from cells of males.

According to still another aspect of the present invention, there is provided a use of the foregoing composition of a polar organic extract of Eurycoma longifolia and/or a fraction derived from the polar organic extract in the manufacture of a medicament for the treatment of male infertility or sexual dysfunction in a human or animal by way of increasing spermatozoa production or count, increasing spermatozoa motility, increasing testosterone synthesis and Increasing testosterone release from cells of males.

The foregoing composition of a polar organic extract of Eurycoma longifolia and/or a fraction derived from the polar organic extract of Eurycoma longifolia can be formulated into various pharmaceutical formulations for clinical use such as capsules including soft gel capsules, tablets, galenicals, powder, granules, aqueous medicine, Injection and the like by standard methods, in which the polar organic extract and/or the fraction thereof is present and administered as active component at an effective therapeutic amount based on its efficacy and toxicity, alone or In combination with other chemicals through various routes of administration such as oral, sublingual, intravenous, intramuscular and the like. The effective amount is sufficient to increase spermatozoa count and spermatozoa quality, i.e. in terms of morphology and motility, as well as to increase testosterone level and testosterone release from cells. The effective amount to improve the male spermatogenesis will depend on the severity of the condition being treated; Individual patient parameters including age, physical condition, size and weight; concurrent treatment and drug interaction; frequency of treatment; and the mode of administration.

Preferably, the adult human doses of the fraction of polar organic extract are from about 0.2-2.0 mg/kg per day. It is expected that the oral doses in the range of 10 to 100 mg per 60 kg adult in one or several administrations per day, will yield the desired results. For the polar organic extract, the adult human oral doses would be in the range of 50 to 500 mg/kg per day. In the event that the response In a subject is insufficient at such doses, higher doses (or effective higher doses by a different, more localized delivery route) may be employed to the extent that the patient tolerance permits. Dose ranges can be adjusted when necessary for the treatment of individual patients and according to the specific condition treated. Multiple doses per day are contemplated to achieve appropriate systemic levels of compounds. The therapy period is on a short-term basis, preferably not more than six (6) months.

### BRIEF DESCRIPTION OF THE DRAWINGS

FIG. 1 is a flowchart illustrating the extraction of the fraction of a polar organic extract of E. longifolia according to the present invention;

FIG. 2 is a flowchart illustrating the isolation scheme of various extracted constituents in the fraction of a polar organic extract of E. longifolia according to the present invention;

FIG. 3 is a flowchart illustrating an experimental design of animal study for 42 days of treatment to assess the fertility properties of the fraction of a polar organic extract of E. longifolia according to the present invention;

FIG. 4 is a flowchart illustrating an experimental design of animal study for the second 42 days without and with treatment to assess the fertility properties of the fraction of polar organic extract of E. longifolia according to the present invention;

FIG. 5 is a graph illustrating the effect on rat sperm count after daily oral administration of the fraction of polar organic extract of E. longifolia at 25.51 mg/kg (E), the extract of A. paniculata at 125 mg/kg (HB) and control (C) for 42 days, and following another 42 days of co-administration of HB+E. WHB and WE are groups not given HB and E, respectively during the second 42 days;

FIG. 6 is a graph illustrating the effect on sperm morphology after an oral administration of the fraction of polar organic extract of E. longifolia at 25.51 mg/kg (E), extract of A. paniculata at 125 mg/kg (HB) and control (C) for 42 days and following another 42 days of co-administration of HB+E. WHB and WE are groups not given HB and E, respectively during the second 42 days;

FIG. 7 is a graph illustrating the effect on sperm motility after an oral administration of the fraction of polar organic extract of E. longifolia at 25.51 mg/kg (E), extract of A. paniculata at 125 mg/kg (HB) and control (C) for 42 days, and following another 42 days of co-administration of HB+E. WHB and WE are groups not given HB and E, respectively during the second 42 days;

FIG. 8 is a graph illustrating the effect on testosterone level per weight of testis after an oral administration of the fraction of polar organic extract of E. longifolia at 25.51 mg/kg (E), extract of A. paniculata at 125 mg/kg (HB) and control (C) for 42 days and following another 42 days of co-administration of HB+E. WHB and WE are groups not given HB and E, respectively during the second 42 days;

FIG. 9 is a graph illustrating the effects on testosterone level in the plasma and testis homogenates of the rats after oral administration of 12.76, 25.51 and 51.02 mg/kg of the fraction of polar organic extract of E. longifolia for 30 days.

FIG. 10 illustrates the histology of testis from the rats of control (C) treated with 10% propylene glycol in water (v/v) (H&E, ×100);

FIG. 11 illustrates the histology of testis from rats treated with the fraction of polar organic extract of E. longifolia at 25.51 mg/kg (H&E, ×100). L, Leydig cells; 1, germ cells; 2, spermatidic cells;

FIG. 12 illustrates the histology of testis from rats treated with A. paniculata extract at 125 mg/kg (H&E, ×100). 3, spermatocyte regression; 4, lumen of seminiferous tubule; and

FIG. 13 illustrates the histology of testis from rats treated with the fraction of polar organic extract of E. longifolia and A. paniculata extract (H&E, ×200).

### DETAILED DESCRIPTION OF THE INVENTION

Various features and aspects of the present invention are illustrated further in the examples that follow. While these examples are presented to show one skilled in the art how to operate within the scope of this invention, they are not to serve as a limitation on the scope of the invention where such scope is only defined in the claims.

### EXAMPLES

### Example 1

### Preparation of the Polar Organic Extract

As for the plant part selected for extraction, the root, the bark or the stem of Eurycoma longifolia Jack (Simaroubaceae family) can be used. Each plant part was dried, shredded into small pieces and then milled before extraction.

The polar organic extract in the present invention is obtained by extracting the plant parts described above with a water-soluble organic solvent that includes methanol, ethanol and acetone or a mixed solution of water and organic solvent thereof. The extraction temperature is adjusted to the range of 50 to 70° C. for 6 to 8 hours per extraction. The extract suspension was filtered and the residue was re-extracted with a fresh solvent as previously described. The amount of organic solvent that may be, for example methanol, ethanol and acetone or a mixed solution of water and organic solvent thereof relative to the dried plant parts is from 1:10 to 1:100. The combined filtrate was evaporated to dryness under partial vacuum at room temperature of 24 to 27° C. to yield 2% to 5% w/w of crude extract.

### Example 2

### Preparation of the Fraction of Polar Organic Extract

The fraction of a polar organic extract of E. longifolia is obtained by separation and purification of the above-mentioned crude extract by chromatography techniques. The polar organic extract is adsorbed onto a styrene-divinylbenzene synthetic resin or a dextran synthetic resin, and then eluted sequentially with water, a mixed solution of water and an organic solvent and followed by an organic solvent. The organic solvent may be a lower alcohol or polar organic solvent, for example, methanol, ethanol, acetone and isopropyl alcohol.

In a preferred embodiment, the resin-adsorbed components of the fraction of polar organic extract are obtained by passing the polar organic extract onto a column packed with a styrene-divinylbenzene synthetic resin or a dextran synthetic resin. Non-adsorbed components are washed off with water and the resin-adsorbed components are eluted with mixed solutions of water and organic solvent, and followed by organic solvent alone.

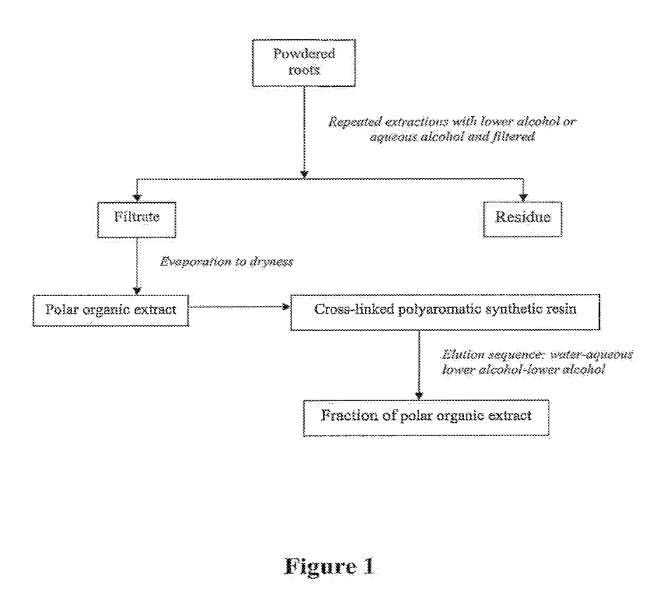

The polar organic solution was concentrated to dryness under partial vacuum to yield 10 to 25% w/w of the desired fraction of E. longifolia. isolation of the fraction of a polar organic extract of E. longifolia is illustrated in FIG. 1.

### Example 3

### Identification of Extracted Components of the Fraction of Polar Organic Extract

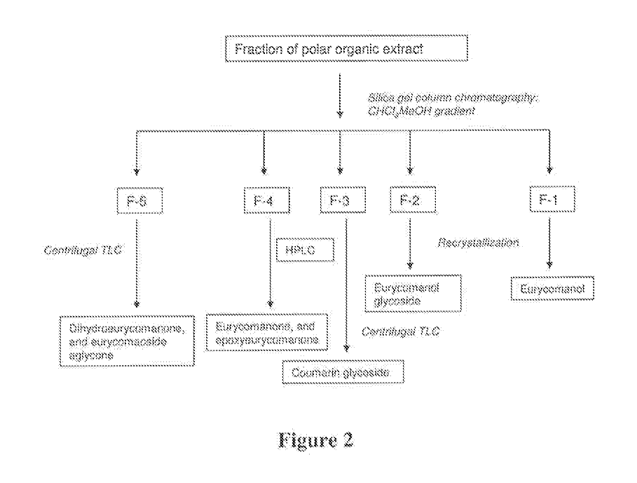

The extracted components in the fraction of polar organic extract of E. longifolia are identified by applying 200 grams of the extract dried residue to a column packed with silica gel and followed by carrying out elution with mixed solutions of chloroform and methanol with increasing polarity. The various sub-fractions displaying similar R₁ value on thin-layer chromatography (TLC) were pooled and combined as F-1 to F-5, as shown in FIG. 2.

Sub-fractions F-1 and F-2 were further purified by repeated methanol re-crystallization to yield eurycomanol ([α]_{D}²⁷: +85.6°) (c 0.31, pyridine) and eurycomanol-2-O-β-glucopyranoside ([α]_{D}²⁷: +78.0°) (c 1.00, pyridine), respectively.

Sub-fraction F-3 was further fractionated by centrifugal TLC with chloroform and increasing concentrations of methanol to yield 6-methoxycoumarin-7-O-α-D-glycopyranoside.

Sub-fraction F-4 was further purified by HPLC using a semi-preparative Partisil 10 ODS-3 5μcolumn (250×7.6 mm). Elution with a mobile phase of acetonitrile:water (1:9) at 1.6 mL min⁻¹ and detection at 210 nm afforded eurycomanone ([α]_{D}²⁷: +34.2°) (c 0.32, pyridine) and 13α(21)-epoxyeurycomanone ([α]_{D}²⁷: +32.1°) (c 0.11, MeOH).

Fractionation of sub-fraction F-5 on centrifugal TLC using mixed solvents of ethyl acetate and increasing methanol concentrations followed by chloroform and increasing concentrations of methanol, afforded eurycomaoside aglycone ([α]_{D}²⁷: +16.4°) (c 0.30, MeOH), and 13,21-dihydroeurycomanone ([α]_{D}²⁷: +46.9°) (c 0.11, MeOH).

Isolation scheme of the various extracted constituents described above is shown in FIG. 2 and their structures were confirmed by comparison of the NMR, MS, UV and IR spectra with those reported (Chan et al., 1986; Morita et al., 1990; Kardono et al., 1991).

### Example 4

### Assessment of Fertility Properties of the Fraction of Polar Organic Extract

To assess the fertility properties of the fraction of polar organic extract of Eurycoma longifolia, male Sprague Dawley (SD) rats, weighing about 220 to 250 g were purchased from the animal house of Universiti Sains Malaysia and maintained on a 12 hour light-dark cycle at ambient room temperature. Animals undergone fasting overnight from food were allowed access to water before the experiments commenced. The experimental protocol was submitted and approved by the Animal Ethics Committee of Universiti Sains Malaysia.

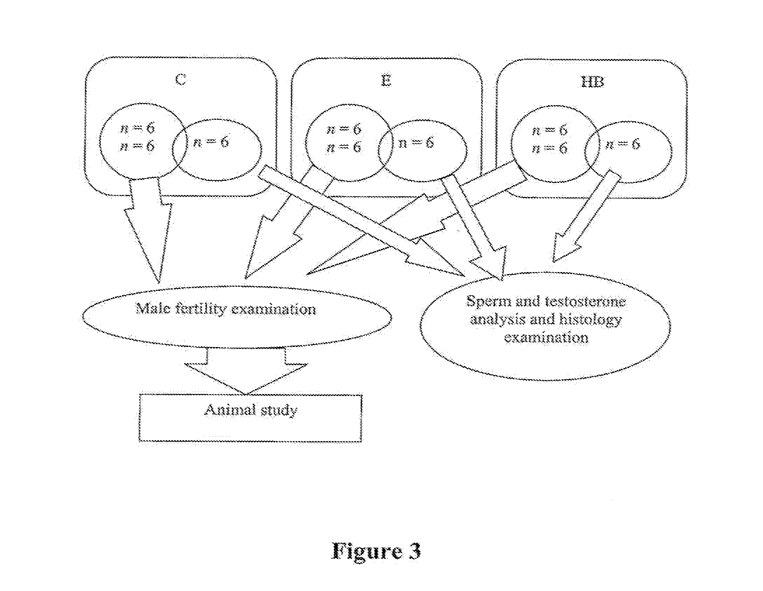

Fifty-four of the rats were divided into three treatment groups consisting of eighteen animals each. The control group (C) animals were given only vehicle containing 10% propylene glycol in water (v/v); those in group E were fed with the fraction of polar organic extract of Eurycoma longifolia at a dose of 25.51 mg/kg in 10% propylene glycol, whereas group HB animals were fed with an extract of Andrographis paniculata at the dose of 125 mg/kg (containing 25 mg/kg of andrographolide). All treatments were administered orally for 42 days consecutively. Subsequently, twelve animals from each group were subjected to male fertility studies involving each male being housed individually for 5 days with two female rats whereby during this period, one estrous cycle has elapsed (Morgan and El-Tawil, 2003). Vaginal swab of all female rats were performed to determine successful copulation. The body weight elevation from the female rats was taken as pregnancy Indicator during the gestation period. Upon birth, the body weight of the pup was recorded on the 4^{th}, 7^{th}, 11^{th}, 14^{th}, 17^{th} and 21^{st} day during lactation period. Fertility and reproductive indices were also determined (Ecobichon, 1995). The other six animals of each group were subjected to sperm analysis, plasma testosterone determination and histological studies. The above experimental design is illustrated in FIG. 3.

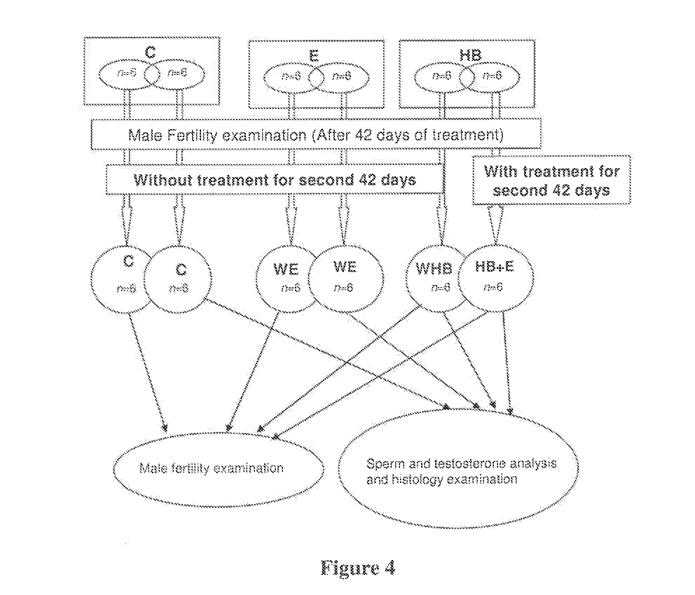

After male fertility studies, the twelve animals from each group of C, E and HB were continued for the next 42 days of study (see FIG. 4). For group E (n=12), the oral administration of the Eurycoma longifolia extract was withdrawn for 42 days as a washout period (WE). In the group HB (n=12), six animals were continuously fed daily with A. paniculata extract at a dose of 125 mg/kg co-administered with the fraction of Eurycoma longifolia at a dose of 25.51 mg/kg for 42 days consecutively (HB+E) whereas, another six animals (WHB) followed a washout period for 42 days. During the washout period, the animals were allowed free access to water and food. At the end of the study, six animals from each group C, WE, HB+E and WHB were subjected to male fertility examination, sperm analysis and testosterone determination. The above experimental design is illustrated in FIG. 4.

### Example 5

### Testosterone and Quassinoid Analysis

Twenty-four male SD rats were divided into four groups. Animals in the control group were given 10% propylene glycol in water (v/v) as vehicle; animals in the other groups were fed with the fraction of Eurycoma longifolia at doses of 12.76, 25.51 and 51.02 mg/kg, respectively. All treatments were administered orally for 30 days consecutively. After 24 hours of the last treatment, all animals were anaesthetized by diethyl ether and 3.0 ml of blood was collected by cardiac puncture for plasma testosterone analysis. They were then sacrificed by an overdose administration of diethyl ether. The testes were removed by orchidectomy (Remie, 2000) for testosterone and quassinoid analysis in the testis homogenates.

### Example 6

### Results

(i) Sperm Analysis

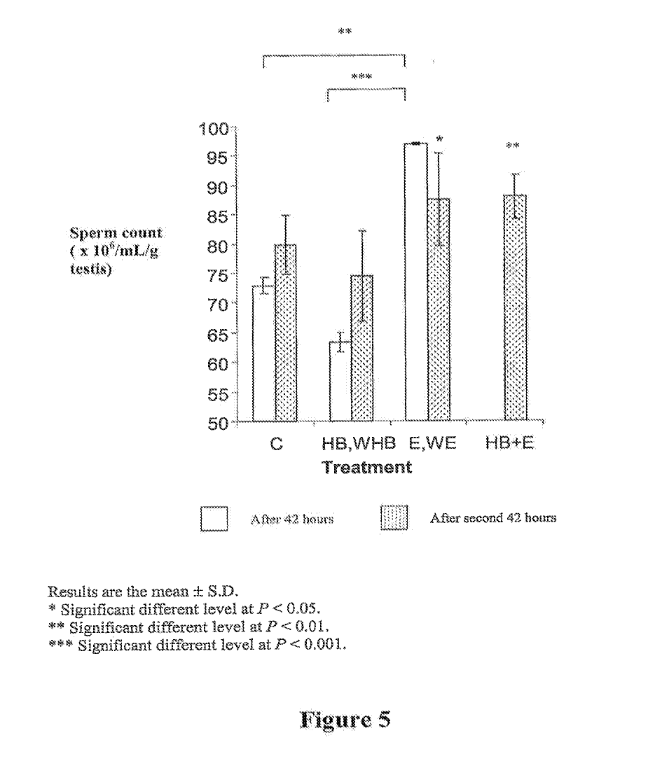

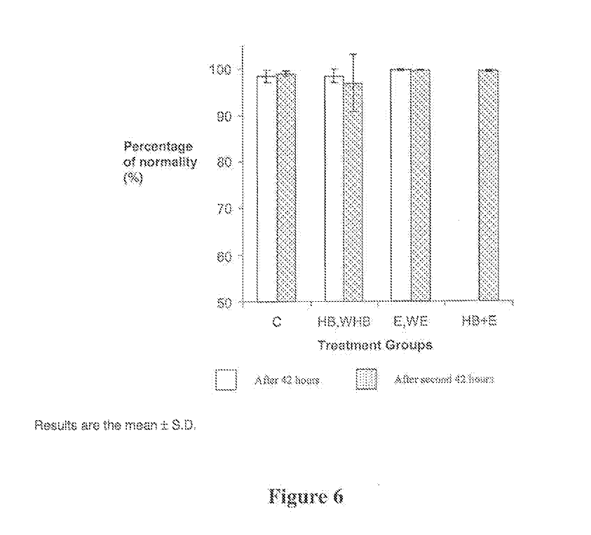

The rats (E) orally administered with the fraction of Eurycoma longifolia, showed a significant increase in sperm count when compared with those from the control C (P<0.01) and A. paniculata extract (HB) (P<0.001) after the first 42 days of treatment (FIG. 5). When treatment of the Eurycoma longifolia fraction and A. paniculata extract was withdrawn, the sperm count of the animals from groups WE and WHB returned to the same level as that of the control (C). Animals (HB+E) that were co-administrated with the Eurycoma longifolia fraction (25.51 mg/kg) and A. paniculata extract (125 mg/kg) displayed an increase of 133.0% in sperm count when compared to those given A. paniculata extract (HB) alone (P<0.01), but was not significantly different from that of WHB and WE (FIG. 5). The sperm count of the animals not given A. paniculata extract (WHB) after the washout period of 42 days showed a slight recovery when compared to the animals under the treatment (HB) (FIG. 5). The sperm count of the animals after withdrawing the Eurycoma longifolia fraction for 42 days during the washout period (WE) was significantly lowered (P<0.05) when compared with the animals given the fraction of E. longifolia (E). Morphology of the sperm from the animals undergoing treatment throughout the 42+42 days of study appeared normal similar to those of the control group, as shown in FIG. 6.

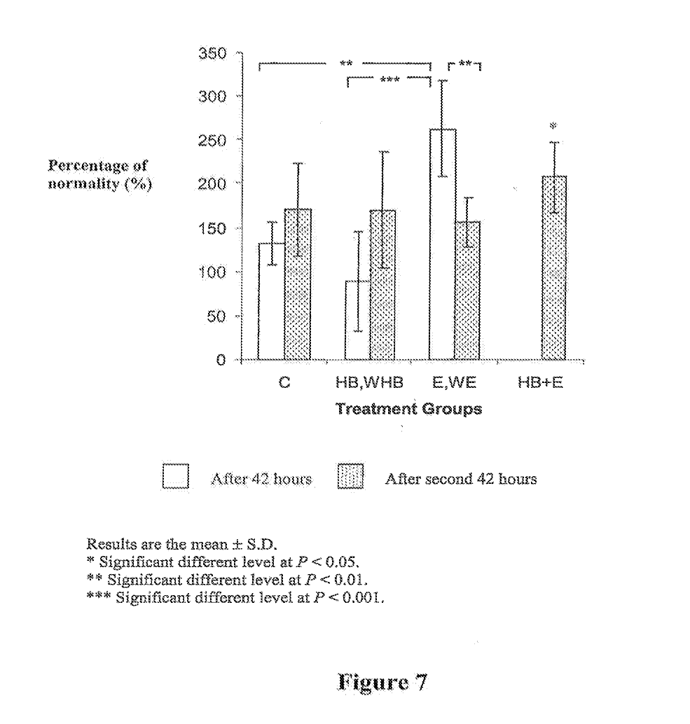

Referring to FIG. 7, sperm motility of the rats treated with the fraction of E. longifolia (E) showed a significant increase when compared with those given A. paniculata extract (HB) (P<0.001) and control (P<0.01) after 42 days of oral administration. Sperm motility of group WE returned to the control level when treatment was withdrawn for 42 days during the washout period. Meanwhile, the rats co-administered with A. paniculata extract and the fraction of E. longifolia (HB+E), displayed significantly higher sperm motility than those of HB (P<0.05). The sperm motility of animals given the extract (E) was significantly higher (P<0.01) than those of WE.

(ii) Testosterone Analysis

Referring to FIG. 8, testosterone level per testicular weight of the rat given the fraction of E. longifolia orally (E) showed an increase when compared with those of the control (C) after 42 days of treatment but was not significantly different when compared with those from group WE and control after 42 days of washout period. However, the hormone levels in the animals of group WE were significantly higher (P<0.05) than that of the animals in group E.

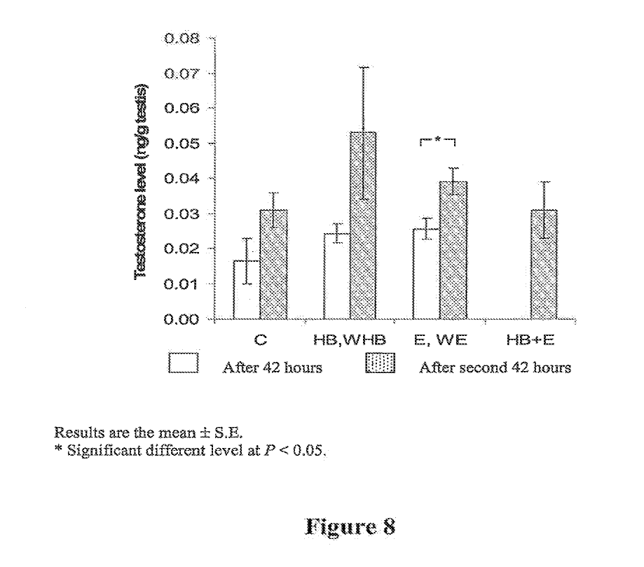

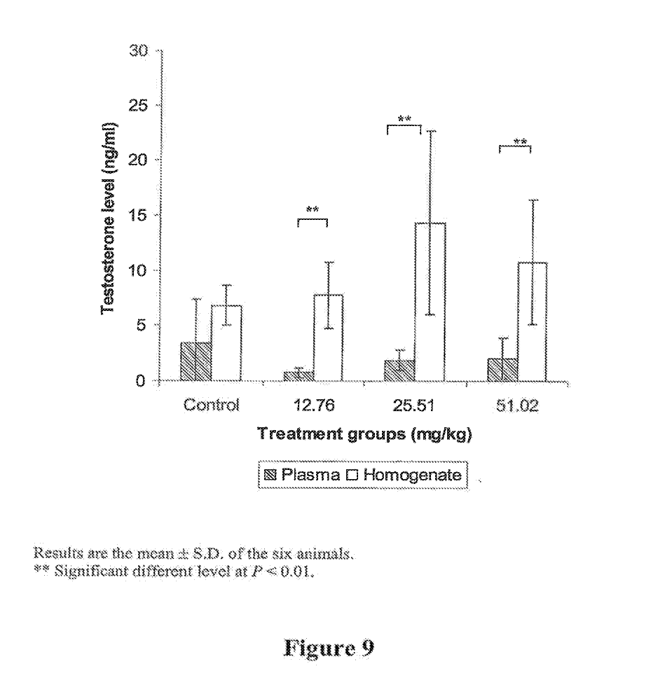

Referring to FIG. 9, comparative studies of the testosterone level in the plasma and the testis were performed after oral administration of 12.76, 25.51 and 51.02 mg/kg of the fraction of E. longifolia for 30 days. The plasma testosterone levels in the control and the treated animals were not significantly different and a typical dose-response of androgen elevation was not observed. In contrast, the testis homogenates showed a significantly higher testosterone level (P<0.01) than the plasma of the animal receiving a dose of 12.76, 25.51 or 51.02 mg/kg of the fraction of E. longifolia whereas, the androgen levels in the plasma and the testis homogenate of the control animal were not significantly different. However, the androgen levels in the testis homogenates at Increasing doses of 12.76, 25.51 and 51.02 mg/kg of the E. longifolia fraction increased but were not significantly different from one another, indicating a dose-response elevation of testosterone was not observed. At 51.02 mg/kg dose, the testosterone level showed a non-significant decrease when compared with that at 25.51 mg/kg, indicating that the dose may be high and a reversal of androgen level was observed. The results indicated that the E. longifolia fraction induced spermatogenesis through an increase of testosterone in the testis of the animals.

(iii) Quassinoid Analysis

HPLC analysis of the testis homogenates of the animals administered orally with 12.76, 25.51 and 51.02 mg/kg of the fraction of E. longifolia detected one of its major quassinoids, eurycomanone and its amount was quantified (Table 1). A calibration curve was plotted using pure eurycomanone Isolated and displayed an equation of Y=1250.3X+43.2 with r²=0.999 [Y: peak height (my); X: eurycomanone (μg/ml)]. The detected amount of eurycomanone in the animal testes showed a correlation of r²=0.972 with increased doses of the E. longifolia fraction. The concentration of eurycomanone may contribute to the increase in testosterone level of the testis homogenates but may not affect its level in the plasma (FIGS. 8 and 9).

TABLE 1
Increasing amount of eurycomanone detected in the rat testis homogenate
after oral administration of the various doses of E. longifolia fraction
for 30 days. Results are the mean ± S.E. of six animals

| Doses (mg/kg) | Eurycomanone concentration (μg/g testis) |
|---|---|
| Control | 0 |
| 12.76 | 1.00 ± 0.41 |
| 25.51 | 1.47 ± 0.33 |
| 51.02 | 1.99 ± 0.23 |

(iv) Histology Examination

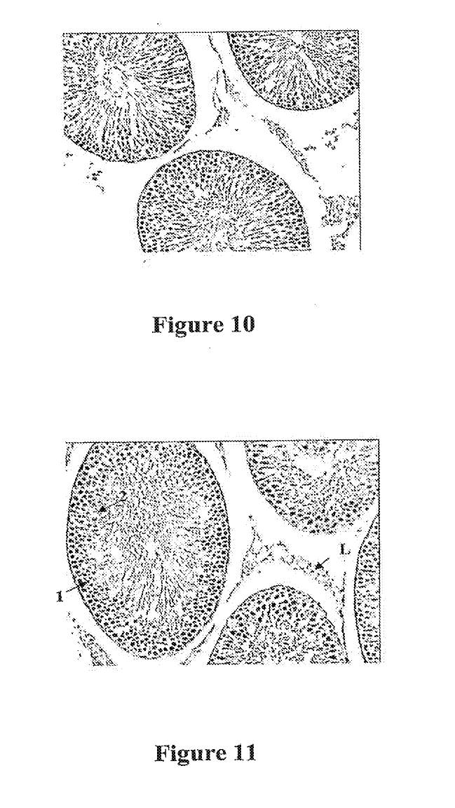

The histology of the rat testis of control C is shown in FIG. 10. The germ layers and spermatocytes in the seminiferous tubules appear normal. The Sertoli cells with elongated cytoplasm extending from the basement membrane to the lumen of the seminiferous tubule are clearly observed.

The histology of the testis of the rat treated with 25.51 mg/kg fraction of E. longifolia (E) is shown in FIG. 11. The germ layers and spermatocytes in the seminiferous tubule are fully intact and crowded when compared with that of control C, indicating proliferation of germ cells 1. The high number of spermatidic cells 2 contributes to the high sperm count. The Leydig cells L are clearly identified In FIG. 11.

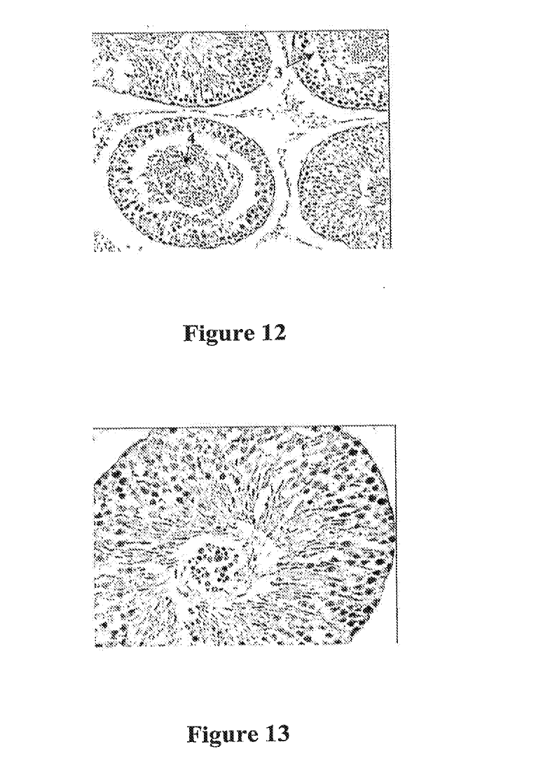

FIG. 12 shows regression 3 of the spermatocytes in the seminiferous tubules of the rat given 125 mg/kg of A. paniculata (HB) orally for 42 days. Prematured separation and shedding of the secondary spermatocytes and spermatids are observed. Matured spermatidic cells are also reduced In the lumen of the tubules 4.

The histology in FIG. 13 shows the testis of a rat initially given orally the extract of A. paniculata at 125 mg/kg for 42 days and subsequently treated with 25.51 mg/kg fraction of E. longifolia (E) forr another 42 days. The adverse effect of A. paniculata on the testis shown in FIG. 12 has been reversed by treatment with E. longifolia. The testis displayed histology approaching that of the control rats.

(v) Male Fertility Examination

The male rats treated with the fraction of E. longifolia (E) showed the highest mating index of 54.17% compared to 37.50% from the control (C) and 31.82% from the group given the extract A. paniculata (HB) (Table 2). Pseudopregnancy or false pregnancy (defined as the absence of menses accompanied by pregnancy symptoms without any conception occurring in the female rats) was observed in the control and treatment groups. Fifty-two pups were born in the control group. All the pups in group E (n=40) and HB (n=26) were alive after birth. The pups in group E showed significantly higher body weight than the control at the early lactation period. In addition, rats treated with the fraction of E. longifolia (E) were more active throughout the lactation and weaning period. Interestingly, about 26 male and 14 female pups were born in the group treated with the fraction of E. longifolia (E) when compared with the sex ratios of those from the control (24 males; 27 females) and HB group (12 males; 14 females).

TABLE 2
Effect of E. longifolia fraction at 25.51 mg/kg (E); the extract of A. paniculata at
125 mg/kg (HB) and control (C) on the fertility of male rats after oral administration
for 42 days.

| | Treatment groups | | |
|---|---|---|---|
| Parameters | C | HB | E |
| Number of male used | 12 | 11 | 12 |
| Number of female used | 24 | 22 | 24 |
| Number of pregnant females | 9 | 7 | 10 |
| Number of female given birth | 6 | 4 | 7 |
| Total number of litters | 52 | 26 | 40 |
| Number of copulation | 9 | 7 | 13 |
| Mean fetal body weight (g) | | | |
| At 4^{th} day after birth | 8.47 ± 0.10 | 9.15 ± 0.22** | 9.29 ± 0.10*** |
| At 7^{th} day after birth | 11.79 ± 0.27 | 12.22 ± 0.38 | 13.08 ± 0.21** |
| At 11^{th} day after birth | 18.99 ± 0.58 | 21.34 ± 0.79* | 22.22 ± 0.54*** |
| At 14^{th} day after birth | 21.85 ± 0.83 | 25.57 ± 1.12* | 25.63 ± 0.96** |
| At 17^{th} day after birth | 25.40 ± 1.05 | 29.56 ± 1.41 | 30.64 ± 1.13** |
| At 21^{st} day after birth | 37.32 ± 1.45 | 44.82 ± 1.48** | 44.04 ± 1.53** |
| Mating index (%) | 37.50 | 31.82 | 54.17 |
| Male fertility index (%) | 75.00 | 63.63 | 83.33 |
| Female fertility index (%) | 37.50 | 31.82 | 41.67 |
| Parturition incidence index (%) | 66.67 | 57.14 | 70.00 |
| Live birth index (%) | 98.08 | 100 | 100 |
| Survival index (%) | 100 | 100 | 100 |
| Litter death index (%) (day 1-4) | 0 | 0 | 0 |
| Litter death index (%) (day 5-21) | 1.96 | 0 | 0 |
| Sex ratio index (%) (at birth) | 92.59 | 85.71 | 185.71 |
| Sex ratio index (%) (day 4, 21) | 88.89 | 85.71 | 185.71 |

Values are expressed as mean ± S.E. for fetal body weight.
*Significantly different at P < 0.05 compared to control.
**Significantly different at P < 0.01 compared to control.
***Significantly different at P < 0.001 compared to control.

After 42 days withdrawal of the E. longifolia fraction, the animals (WE) displayed slightly higher mating index (50.00%) and male fertility index (83.33%) (Table 3) than those treated animals (E) (Table 2). Pseudopregnancy from the female rats in the control and treatment groups was also observed. The body weight of the pups at the 21^{st} day after birth derived from HB+E treated animals was significantly less compared with those from the control (P<0.05). The sex ratio of the pups from WE (14 males; 13 females) was significantly different (P<0.05) from the sex ratio of the pups from E (26 males; 14 females, Table 2). No birth was observed from the animals (WHB) even though treatment of A. paniculata extract was discontinued for 42 days. The male fertility index of the animals In group WHB dropped to 40.00% (Table 3) when compared to 63.63% of HB in Table 2.

TABLE 3
Effect on the fertility of male rats after discontinuation of treatment with A. paniculata
extract (WHB); E. longifolia fraction (WE) and continuation of treatment with
A. paniculata extract (125 mg/kg) and E. longifolia fraction (25.51 mg/kg) (HB + E) for
another 42 days

| | Treatment groups | | | |
|---|---|---|---|---|
| Parameters | Control | WHB | WE | HB + E |
| Number of male used | 6 | 5 | 6 | 6 |
| Number of female used | 12 | 10 | 12 | 12 |
| Number of pregnant females | 3 | 2 | 5 | 4 |
| Number of female given birth | 2 | 0 | 4 | 3 |
| Total number of litters | 15 | 0 | 27 | 30 |
| Number of copulation | 5 | 4 | 6 | 5 |
| Mean fetal body weight (g) | | | | |
| At 4^{th} day after birth | 11.21 ± 0.21 | — | 9.63 ± 0.32* | 10.18 ± 0.35 |
| At 7^{th} day after birth | 15.37 ± 0.41 | — | 13.42 ± 0.51 | 13.88 ± 0.52 |
| At 11^{th} day after birth | 21.66 ± 0.57 | — | 20.05 ± 0.85 | 19.47 ± 0.66 |
| At 14^{th} day after birth | 26.39 ± 0.59 | — | 24.89 ± 1.04 | 23.20 ± 0.73 |
| At 17^{th} day after birth | 30.24 ± 0.74 | — | 30.40 ± 1.26 | 27.02 ± 0.94 |
| At 21^{st} day after birth | 46.07 ± 1.22 | — | 45.47 ± 1.52 | 39.20 ± 1.09* |
| Mating index (%) | 41.67 | 40.00 | 50.00 | 41.67 |
| Male fertility index (%) | 50.00 | 40.00 | 83.33 | 66.67 |
| Female fertility index (%) | 25.00 | 20.00 | 41.67 | 33.33 |
| Parturition incidence index (%) | 66.67 | 0 | 80.00 | 75.00 |
| Live birth index (%) | 80.00 | 0 | 100 | 96.67 |
| Survival index (%) | 100 | 0 | 100 | 100 |
| Litter death index (%) (day 1-4) | 25.00 | 0 | 0 | 0 |
| Litter death index (%) (day 5-21) | 0 | 0 | 0 | 3.45 |
| Sex ratio index (%) (at birth) | 87.50 | — | 107.69 | 150.00 |
| Sex ratio index (%) (day 4, 21) | 50.00 | — | 107.69^{@} | 163.64 |

Values are expressed as mean ± S.E. for fetal body weight.
*Significantly different at P < 0.05 compared to control.
**Significantly different at P < 0.01 compared to control.

1-16. (canceled)

17. A method of isolating at least one bioactive component of Eurycoma longifolia the method comprising the steps of:
(i) preparing at least one polar organic extract from Eurycoma longifolia plant or part thereof; and
(ii) passing the polar organic extract through at least one cross-linked polyaromatic synthetic resin to obtain the bioactive component of Eurycoma longifolia.

18. The method according to claim 17, wherein the resin is at least one styrene-divinylbenzene synthetic resin and/or at least one dextran synthetic resin.

19. The method according to claim 17, wherein the method further comprises a step of eluting the bioactive component from the resin sequentially with water, at least one mixed solution of water and at least one organic solvent, and at least one organic solvent to produce at least one elute.

20. The method according to claim 19, wherein the organic solvent is at least one lower alcohol or at least one polar organic solvent.

21. The method according to claim 19, wherein the organic solvent is selected from the group consisting of methanol, ethanol, acetone and isopropyl alcohol.

22. The method according to claim 17, further comprising a step of purifying the bioactive component using at least one method selected from the group consisting of partitioning, chromatography and recrystallization.

23. The method according to claim 17, wherein step (i) involves subjecting pulverized roots, barks or stems of Eurycoma longifolia for extraction with at least one organic solvent or at least one mixed solution of water and organic solvent thereof.

24. The method according to claim 19, wherein the elute comprises at least 10% to 25% by weight of the bioactive component of Eurycoma longifolia.

25. A method according to claim 17, wherein the bioactive agent is at least one quassinoid and/or coumarin.

26. The method according to claim 25, wherein the quassinoid is selected from the group consisting of eurycomanone, 13α,21-dihydroeurycomanone, 13(21)-epoxyeurycomanone, eurycomanol, glycoside of eurycomanol, eurycomaoside, and aglycone of eurycomaoside; and/or coumarin is selected from the group consisting of, 6-methoxycoumarin-7-O-α-D-glycopyranoside, and glycoside of 6-methoxycoumarin-7-O-α-D-glycopyranoside.

27. The method according to claim 17, wherein the bioactive agent has a bioactivity of increasing spermatozoa count, increasing spermatozoa motility, and/or increasing testosterone synthesis and release from cells.
